(19) United States
(12) Patent Application Publication
(10) Pub. No.: US 2026/0011399 A1
(43) Pub. Date: Jan. 8, 2026

(54) MODEL-BASED PEPTIDE DESIGN METHOD

(71) Applicant: BEIJING BAIDU NETCOM SCIENCE TECHNOLOGY CO., LTD., Beijing (CN)

(72) Inventors: Yang XUE, Beijing (CN); Xiaomin FANG, Beijing (CN); Xiaonan ZHANG, Beijing (CN)

(73) Assignee: BEIJING BAIDU NETCOM SCIENCE TECHNOLOGY CO., LTD., Beijing (CN)

(21) Appl. No.: 19/326,253

(22) Filed: Sep. 11, 2025

(30) Foreign Application Priority Data
Jun. 3, 2025 (CN) 202510733056.6

(51) Int. Cl. G16B 15/30; G16B 15/20; G16B 40/20

(52) U.S. Cl. CPC G16B 15/30; G16B 15/20; G16B 40/20

(57) ABSTRACT

A model-based peptide design method in the field of artificial intelligence technology such as biological computing is provided. The specific implementation includes: obtaining a pocket of an objective target protein and an objective peptide, a reserved position for designing a unnatural amino acid is identified in the objective peptide, and the pocket binds to the objective peptide via the reserved position; obtaining a feature of the pocket of the objective target protein and multimodal features of each known amino acid in the objective peptide; the multimodal features of each known amino acid comprise a backbone orientation feature, a backbone rotation feature, a side chain type feature, and a rigid atom group distribution feature; designing the unnatural amino acid at the reserved position in the objective peptide using a pre-trained peptide design model based on the feature of the pocket of the objective target protein and the multimodal features of each known amino acid in the objective peptide.

### CROSS-REFERENCE TO RELATED APPLICATION

The present disclosure claims the priority and benefit of Chinese Patent Application No. 202510733056.6, filed on Jun. 3, 2025. The disclosure of the above application is incorporated herein by reference in its entirety.

### TECHNICAL FIELD

The present application relates to the field of computer technology, particularly to the field of artificial intelligence technology such as biological computing, and more particularly to a model-based peptide design method.

### BACKGROUND

Peptide design with unnatural amino acids is a cutting-edge technology that enhances pharmacological properties and functional diversity by incorporating artificially synthesized or chemically modified unnatural amino acids into peptide chains, breaking through the structural limitations of natural amino acids.

As bioactive molecules composed of 3-30 amino acids, peptides play crucial roles in life processes such as signal transduction and immune regulation through specific recognition of target protein receptors.

### SUMMARY

According to an embodiment of the present disclosure, a model-based peptide design method is provided, which includes:
obtaining a pocket of an objective target protein and an objective peptide, where a reserved position for designing an unnatural amino acid is identified in the objective peptide; and the pocket of the objective target protein binds to the objective peptide via the reserved position;
obtaining a feature of the pocket of the objective target protein and multimodal features of each known amino acid in the objective peptide; where the multimodal features of each known amino acid includes a backbone orientation feature, a backbone rotation feature, a side chain type feature, and a rigid atom group distribution feature; and
designing the unnatural amino acid at the reserved position in the objective peptide using a pre-trained peptide design model based on the feature of the pocket of the objective target protein and the multimodal features of each known amino acid in the objective peptide.

According to an embodiment of the present disclosure, the peptide design model is trained by:
obtaining a pocket of a training target protein and a training peptide, where the training peptide binds to the training target protein via an unnatural amino acid at a preset position;
obtaining a feature of the pocket of the training target protein and first multimodal features of an amino acid at each position in the training peptide; where the first multimodal features of the amino acid at each position include a backbone orientation feature, a backbone rotation feature, a side chain type feature, and a rigid atom group distribution feature;
training the peptide design model based on the feature of the pocket of the training target protein and the first multimodal features of the amino acid at each position in the training peptide.

According to an embodiment of the present disclosure, an electronic device is provided, which includes:
at least one processor; and
a memory communicatively connected to the at least one processor; where
the memory stores instructions executable by the at least one processor to cause the at least one processor to perform the method of the above-mentioned aspect and any possible implementation.

According to an embodiment of the present disclosure, a non-transitory computer-readable storage medium storing computer instructions is provided, where the computer instructions are used to cause the computer to perform the method of the above-mentioned aspect and any possible implementation.

It should be understood that the contents described in this section are not intended to identify key or essential features of the embodiments of the present disclosure, nor are they used to limit the scope of the present disclosure. Other features of the present disclosure will become readily understood through the following specification.

### BRIEF DESCRIPTION OF THE DRAWINGS

The drawings are used for a better understanding of a solution and do not constitute limitations of the present application. In the drawings,

FIG. 1 is a schematic diagram according to a first embodiment of the present disclosure;

FIG. 2 is a schematic diagram according to a second embodiment of the present disclosure;

FIG. 3 is a schematic diagram showing the principle of a peptide design model provided by the present disclosure;

FIG. 4 is a schematic diagram according to a third embodiment of the present disclosure;

FIG. 5 is a schematic diagram according to a fourth embodiment of the present disclosure;

FIG. 6 is a schematic diagram according to a fifth embodiment of the present disclosure;

FIG. 7 is a schematic diagram according to a sixth embodiment of the present disclosure;

FIG. 8 is a schematic diagram according to a seventh embodiment of the present disclosure;

FIG. 9 is a schematic diagram according to a eighth embodiment of the present disclosure;

FIG. 10 is a schematic diagram according to a ninth embodiment of the present disclosure; and

FIG. 11 is a block diagram of an electronic device for implementing the method of the embodiments of the present disclosure.

### DETAILED DESCRIPTION OF EMBODIMENTS

The following description of exemplary embodiments of the present disclosure is made with reference to the accompanying drawings, which includes various details of the embodiments to aid in understanding, and should be considered merely exemplary. Therefore, those skilled in the art should recognize that various changes and modifications can be made to the embodiments described herein without departing from the scope and spirit of the present disclosure. Similarly, descriptions of known functions and structures are omitted for clarity and conciseness.

Apparently, the described embodiments are only a part of the embodiments of the present disclosure, not all of them. All other embodiments obtained by those skilled in the art without creative work based on the embodiments of the present disclosure fall within the protection scope of the present disclosure.

It should be noted that the terminal device involved in the embodiments of the present disclosure may include but are not limited to a mobile phone, a Personal Digital Assistants (PDA), a wireless handheld device, a Tablet Computers and other smart device; The display device may include but is not limited to a personal computer, a television and other device with display function.

Additionally, the term “and/or” herein is merely a description of the associative relationship of associative objects, indicating that three relationships can exist. For example, A and/or B can indicate: A exists alone, both A and B exist simultaneously, or B exists alone. Furthermore, the character “/” herein generally indicates an “or” relationship between the associated objects before and after it.

In practical applications, although peptide drugs show unique advantages in the Hit-To-Lead stage of drug development, natural peptides generally face druggability bottlenecks such as poor membrane permeability and low metabolic stability. The introduction of unnatural amino acids provides a systematic solution: enhancing a-helix rigidity through cyclic topological structures, extending plasma half-life through hydrophobic side chain modifications, and improving target binding specificity through functional group grafting, achieving precise regulation from molecular structure to biological function.

In existing technology, the chemical diversity of unnatural amino acids, such as chiral centers and non-standard functional groups, makes their structural compatibility more complex than natural amino acids. Existing peptide design schemes can only capture the contextual molecular features of amino acids in a peptide sequence and cannot perform accurate and effective peptide design.

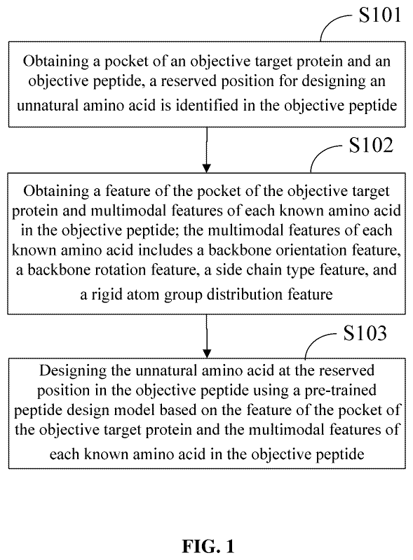

FIG. 1 is a schematic diagram according to a first embodiment of the present disclosure. As shown in FIG. 1, this embodiment provides a model-based peptide design method, which specifically includes the following steps:
S101: Obtaining a pocket of an objective target protein and an objective peptide, a reserved position for designing an unnatural amino acid is identified in the objective peptide;

Specifically, the objective peptide is the peptide to be designed in this embodiment, and the objective target protein is a target protein whose binding performance with the objective peptide is to be studied in the scenario of this embodiment. The pocket of the objective target protein binds to the objective peptide via the reserved position;

Specifically, the obtained pocket of the objective target protein may include the structure of the pocket portion of the objective target protein. Correspondingly, the obtained objective peptide also includes the amino acid structures at other positions on the peptide chain except for the reserved position.
S102: Obtaining a feature of the pocket of the objective target protein and multimodal features of each known amino acid in the objective peptide; the multimodal features of each known amino acid includes a backbone orientation feature, a backbone rotation feature, a side chain type feature, and a rigid atom group distribution feature;

Specifically, the feature of the pocket of the objective target protein may be a digitalized feature of the structure of the pocket of the objective target protein using appropriate molecular description language, which may be in the form of a vector.

In this embodiment, the obtained multimodal features of each known amino acid in the objective peptide include four modal structural features: a backbone orientation feature, a backbone rotation feature, a side chain type feature, and a rigid atom group distribution feature. In practical applications, the multimodal features of each known amino acid may also include other secondary structural features or tertiary structural features of the objective peptide, which will not be elaborated here. For example, the secondary structural features of the objective peptide may include features related to the folding types of different segments in the peptide sequence, alpha helices, beta sheets, etc. The tertiary structural features of the objective peptide include features related to the three-dimensional coordinates of atoms in the amino acids in the peptide sequence.

Among them, the backbone orientation feature, also called the backbone 0 direction feature, may be in a vector form, representing the residue spatial positioning relative to the global coordinates of the objective peptide, using the CA (i.e., the C at the α position of the current amino acid) atom as the reference point in Cartesian coordinates. The backbone rotation feature, also called the backbone rotation matrix, may be specifically characterized by the rotation parameters of the local coordinate system constructed by the N-CA-C atoms of the current amino acid relative to the global coordinate system of the objective peptide, describing the spatial orientation of residues. The side chain type feature represents the side chain type of the current amino acid, which is a discrete classification feature containing a natural or an unnatural amino acid. The rigid atom group distribution feature specifically divides the current amino acid side chain into several rigid atom groups; within each rigid atom group, motion-correlated atoms in the current amino acid side chain are clustered together. In practical scenarios, motion-correlated atoms can be considered as atoms located on the same plane in the structure of the current amino acid.

The multimodal features of each known amino acid in the objective peptide obtained in this embodiment can accurately and effectively characterize the detailed structural information of each amino acid in the objective peptide, providing effective support for designing the unnatural amino acid at the reserved position in the objective peptide.
S103: Designing the unnatural amino acid at the reserved position in the objective peptide using a pre-trained peptide design model based on the feature of the pocket of the objective target protein and the multimodal features of each known amino acid in the objective peptide.

Specifically, during use, the feature of the pocket of the objective target protein and the multimodal features of each known amino acid in the objective peptide can be input into a pre-trained peptide design model. Based on the input information, the peptide design model can accurately and effectively predict the unnatural amino acid at the reserved position, achieving accurate and effective design of the objective peptide.

The model-based peptide design method of this embodiment can accurately and effectively design the unnatural amino acid at the reserved position in the objective peptide using a pre-trained peptide design model based on the feature of the pocket of the objective target protein and the multimodal features of each known amino acid in the objective peptide, achieving the design of the objective peptide. Since the utilized multimodal features of each known amino acid in the objective peptide carry detailed tertiary structural information of each amino acid in the objective peptide, it can effectively improve the accuracy of the designed unnatural amino acid at the reserved position in the objective peptide.

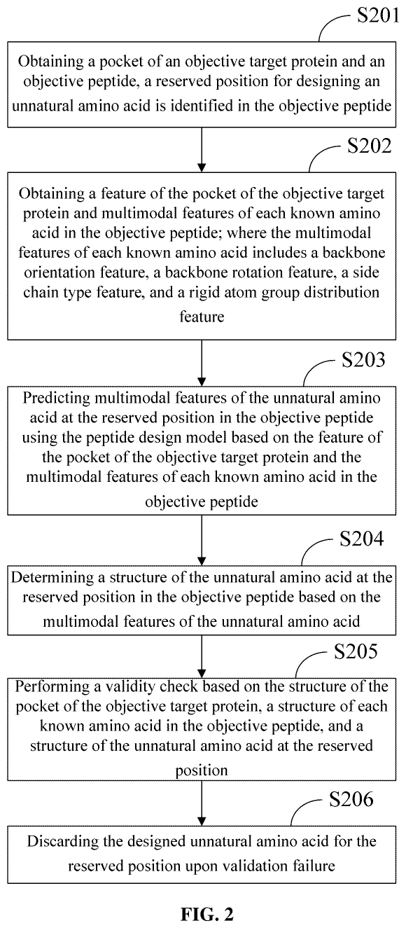

FIG. 2 is a schematic diagram according to a second embodiment of the present disclosure.

The model-based peptide design method of this embodiment, based on the technical solution of the embodiment shown in FIG. 1, further describes the technical solution of the present disclosure in more detail.

As shown in FIG. 2, the model-based peptide design method of this embodiment specifically includes the following steps:

S201: Obtaining a pocket of an objective target protein and an objective peptide, a reserved position for designing an unnatural amino acid is identified in the objective peptide;

In this embodiment, the amino acids at positions other than the reserved position in the objective peptide may be natural amino acids or verified unnatural amino acids. For example, currently common natural amino acids may include 20 types, and verified unnatural amino acids may include 160 types. Of course, with the development of medical technology, the number of verified unnatural amino acids will continue to increase.

S202: Obtaining a feature of the pocket of the objective target protein and multimodal features of each known amino acid in the objective peptide; where the multimodal features of each known amino acid includes a backbone orientation feature, a backbone rotation feature, a side chain type feature, and a rigid atom group distribution feature;

In this embodiment, when obtaining the rigid atom group distribution feature, several rigid atom groups are divided in the current amino acid side chain based on the dependence relationship between each atom and dihedral angles in the current amino acid side chain. Specifically, during division, by analyzing the coupling of side chain atoms during dihedral angle rotation, motion-correlated atoms are clustered into the same rigid unit, thereby establishing a quantifiable rigid atom group distribution. Here, motion-correlated atoms may also be considered as atoms located on the same plane. For example, in specific implementation, atoms may be analyzed sequentially through traversal according to the order from near to far from the main chain of atoms on the side chain, dividing atoms located on the same plane, i.e., motion-correlated atoms, into the same rigid unit, achieving the division of rigid atom groups corresponding to the current amino acid. Even for unnatural amino acids with complex side chains, using this method can accurately and efficiently obtain corresponding rigid atom group distribution features.

The side chain type feature in this embodiment is a discretized feature. As mentioned in the above embodiment, this side chain type may be any one of 20 natural amino acids or any one of 160 verified unnatural amino acids. The side chain type feature may be represented by the identifier of the corresponding type of amino acid, where each amino acid identifier uniquely represents the corresponding amino acid. S203: Predicting multimodal features of the unnatural amino acid at the reserved position in the objective peptide using the peptide design model based on the feature of the pocket of the objective target protein and the multimodal features of each known amino acid in the objective peptide;

For example, in specific implementation of this step, the multimodal features of the unnatural amino acid at the reserved position in the objective peptide may be predicted accurately and effectively using the peptide design model by referring to pre-obtained initial features of the unnatural amino acid at the reserved position, based on the feature of the pocket of the objective target protein and the multimodal features of each known amino acid in the objective peptide.

Specifically, before the implementation of this step, it may also include the following steps:
Obtaining an initial backbone orientation feature of the unnatural amino acid at the reserved position in the objective peptide based on a Gaussian distribution such as [symbol] (0,I₃);
Obtaining an initial backbone rotation feature of the unnatural amino acid at the reserved position in the objective peptide based on a uniform distribution such as U(SO(3));
Obtaining an initial side chain type feature of the unnatural amino acid at the reserved position in the objective peptide based on a Gaussian distribution such as [symbol] (0,K²I);
Obtaining an initial rigid atom group distribution feature of the unnatural amino acid at the reserved position in the objective peptide based on a uniform distribution such as U([0,2π)⁵).

The peptide design model in this embodiment may be a conditional flow matching design model, where the initial features of the unnatural amino acid at the reserved position in the objective peptide can serve as noise input. Based on the input feature of the pocket of the objective target protein and the multimodal features of known amino acids at positions other than the reserved position in the objective peptide, the peptide design model performs denoising and recovery on the input noise, decoding to obtain the multimodal features of the unnatural amino acid at the reserved position in the objective peptide, which may include, for example, the backbone orientation feature, backbone rotation feature, side chain type feature, and rigid atom group distribution feature of the unnatural amino acid at the reserved position.

The design principle of the peptide design model in this embodiment may be considered as using a Conditional Flow Matching to achieve the prediction of four modalities for the unnatural amino acid at the reserved position from an initial distribution to a target distribution. Among them: the initial distribution of the backbone orientation feature, i.e., the initial backbone orientation feature, follows a Gaussian distribution [symbol] (0,I₃); the initial distribution of the backbone rotation feature, i.e., the initial backbone rotation feature, follows a uniform distribution U(SO(3)); the initial distribution of the side chain type feature, i.e., the initial side chain type feature, follows a Gaussian distribution [symbol] (0,K²I); and the initial distribution of the rigid atom group distribution feature, i.e., the initial rigid atom group distribution feature, follows a uniform distribution U([0,2π)⁵). By adopting the above method, the set initial features of the unnatural amino acid at the reserved position are very reasonable and accurate, providing an effective foundation for the subsequent denoising and decoding process to accurately predict the multimodal features of the unnatural amino acid at the reserved position.

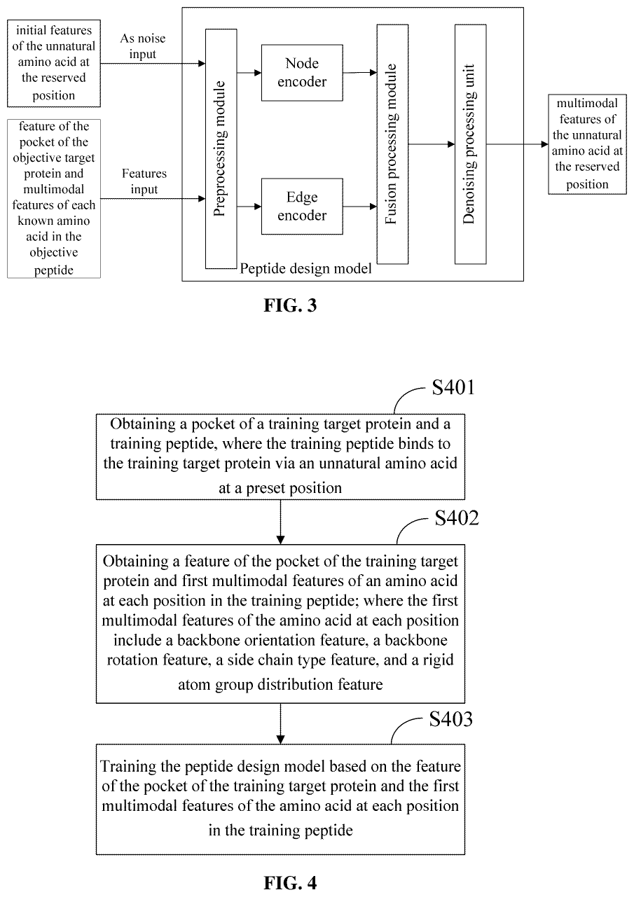

FIG. 3 is a schematic diagram showing the principle of a peptide design model provided by the present disclosure. As shown in FIG. 3, the peptide design model may include a preprocessing module, a node encoder, an edge encoder, a fusion processing module, and a denoising processing module. The node encoder and edge encoder can respectively extract features of individual amino acids and features between adjacent amino acids. For example, the preprocessing module can process the input information to obtain information required by the node encoder and edge encoder, and input them to the node encoder or edge encoder respectively. For each amino acid, the node encoder can generate residue-level node embeddings by fusing amino acid type features, local atomic coordinate features, and backbone dihedral angle features. The local atomic coordinate features can be obtained by the preprocessing module based on the current amino acid type, retrieving the local atomic coordinates of that amino acid from a pre-collected amino acid information library, and thus obtaining the local atomic coordinate features of that amino acid. The amino acid information library may include information of all natural amino acids and all verified unnatural amino acids, for example, it may include local coordinate information of various atoms in each amino acid in Cartesian coordinates with the CA atom as the reference point. For each amino acid, the preprocessing module can also obtain the backbone dihedral angle information of the current amino acid based on its local coordinate information. For the information of the amino acid at the reserved position, corresponding processing can be performed based on the input initial features of the unnatural amino acid at that reserved position.

Specifically, in the node encoder: the amino acid type is encoded by an embedding layer, the local atomic coordinates are expanded into high-dimensional features according to amino acid type after local coordinate system transformation, and the • backbone dihedral angles use angle encoding. The three types of features are concatenated and fused through multilayer perceptron (MLP), and information leakage is controlled through multiple masks such as sequence masks, structure masks, and residue masks, and finally a feature vector for each residue is output. This can effectively handle the integration of protein sequence and structural information.

Edge encoding focuses on modeling relationships between residues. Amino acid pairs represent type interactions through pair embedding, relative position embedding encodes sequence distances. Inter-atomic distances are transformed through Gaussian kernels and MLP to generate distance embedding, achieving dynamic weighting through learnable distance coefficients. At the same time, dihedral angles of paired residues capture spatial orientation correlations through angle encoding. All features are output as edge embeddings through a fusion network, comprehensively characterizing structural adjacency, sequence correlation, and spatial interactions between residues. The edges between adjacent amino acids in the peptide can be predetermined or predetermined by the preprocessing module based on a detection strategy. For example, two amino acids are considered to have an edge if their closest atom pair distance is less than 4 Å. Similarly, edges between the pocket of the objective target protein and the unnatural amino acid at the reserved position in the objective peptide can be detected in the preprocessing module. There can be one, two, or multiple edges between the pocket and the unnatural amino acid at the reserved position.

As shown in FIG. 3, the fusion processing module fuses all feature information encoded by the node encoder and edge encoder, and the denoising processing module performs denoising decoding based on the fused information to recover and output the multimodal features of the unnatural amino acid at the preset position.

Based on the working principle of the above peptide design model, the peptide design model is a conditional flow matching model implemented based on graph neural networks. The initial features of the unnatural amino acid at the reserved position in the objective peptide serve as noise input to the peptide design model, and the peptide design model decodes and recovers the noise based on other input features to obtain the multimodal features of the unnatural amino acid at that reserved position.
S204: Determining a structure of the unnatural amino acid at the reserved position in the objective peptide based on the multimodal features of the unnatural amino acid;

Specifically, based on the multimodal features of the unnatural amino acid at the reserved position predicted by the peptide design model, and combined with information such as bond lengths and bond angles possessed by chemical molecules, the coordinates of each atom in the unnatural amino acid at that reserved position can be determined, thereby obtaining the structure of that unnatural amino acid.
S205: Performing a validity check based on the structure of the pocket of the objective target protein, a structure of each known amino acid in the objective peptide, and a structure of the unnatural amino acid at the reserved position;
S206: Discarding the designed unnatural amino acid for the reserved position upon validation failure.

The validity check in this embodiment can be considered as atomic collision detection. For example, given the structure of the pocket of the objective target protein and the structure of each known amino acid and the unnatural amino acid at the reserved position in the designed objective peptide, the coordinates of atoms in the pocket structure and the coordinates of atoms in the amino acid structure at each position in the objective peptide can be obtained. Then, atomic collisions can be further detected; specifically, this means checking whether there are collisions between each atom in the designed unnatural amino acid at the reserved position and each atom in both the pocket structure and other amino acids in the objective peptide. If collisions exist, the designed unnatural amino acid for the reserved position in the objective peptide is considered invalid and should be discarded. If the check result is valid, the designed unnatural amino acid for the reserved position is retained, thereby achieving the design of the objective peptide.

The model-based peptide design method of this embodiment can accurately and effectively achieve the design of unnatural amino acids in peptides, and thus accurately and effectively achieve peptide design, by predicting the multimodal features of the unnatural amino acid at the reserved position using the peptide design model based on the feature of the pocket of the objective target protein and the multimodal features of each known amino acid in the objective peptide, and then determining the structure of the unnatural amino acid at the reserved position in the objective peptide. Since the multimodal features carry rich internal structural information of amino acids, adopting the technical solution of this embodiment can achieve accurate and effective design.

Furthermore, the technical solution can also perform validity check based on the structure of the pocket of the objective target protein, the structure of each known amino acid in the objective peptide, and the structure of the unnatural amino acid at the reserved position, to remove invalid peptides and ensure the accuracy and effectiveness of peptides for subsequent research and experimental phases.

Through experimental verification, peptides designed using this technical solution can effectively improve affinity; meanwhile, pharmacochemical properties such as stability and half-life can also be improved, making them more suitable for advancing to the next clinical development phase.

The technical solution of this embodiment can be widely applied in G Protein-Coupled Receptors (GPCR) targeted drug development, as well as anti-infection and antibacterial treatment fields.

FIG. 4 is a schematic diagram according to a third embodiment of the present disclosure. As shown in FIG. 4, the training method for the peptide design model in this embodiment specifically includes the following steps:

S401: Obtaining a pocket of a training target protein and a training peptide, where the training peptide binds to the training target protein via an unnatural amino acid at a preset position;

It should be noted that the training peptide also includes amino acids at multiple other positions.

S402: Obtaining a feature of the pocket of the training target protein and first multimodal features of an amino acid at each position in the training peptide; where the first multimodal features of the amino acid at each position include a backbone orientation feature, a backbone rotation feature, a side chain type feature, and a rigid atom group distribution feature;

The specific implementation method of this step can refer to the relevant descriptions in the embodiments shown in FIG. 1 or FIG. 2 above, which will not be repeated here.

S403: Training the peptide design model based on the feature of the pocket of the training target protein and the first multimodal features of the amino acid at each position in the training peptide.

The training method for the peptide design model in this embodiment can effectively improve the accuracy of the trained peptide design model by training the peptide design model based on the feature of the pocket of the training target protein and the first multimodal features of the amino acid at each position in the training peptide, since the multimodal features carry rich internal structural information of amino acids.

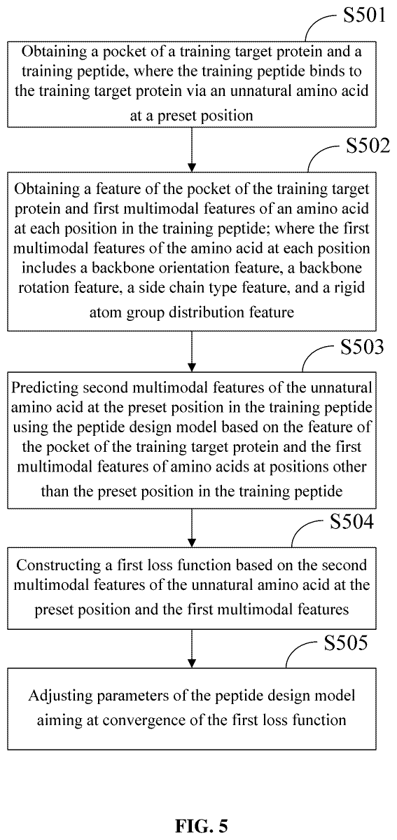

FIG. 5 is a schematic diagram according to a fourth embodiment of the present disclosure. The training method for the peptide design model in this embodiment further describes the technical solution of the present disclosure in more detail based on the technical solution of the embodiment shown in FIG. 4. As shown in FIG. 5, the training method specifically includes the following steps:

S501: Obtaining a pocket of a training target protein and a training peptide, where the training peptide binds to the training target protein via an unnatural amino acid at a preset position;

S502: Obtaining a feature of the pocket of the training target protein and first multimodal features of an amino acid at each position in the training peptide; where the first multimodal features of the amino acid at each position includes a backbone orientation feature, a backbone rotation feature, a side chain type feature, and a rigid atom group distribution feature;

The specific implementation can refer to step S102 in the embodiment shown in FIG. 1 or step S202 in the embodiment shown in FIG. 2, which will not be repeated here.

S503: Predicting second multimodal features of the unnatural amino acid at the preset position in the training peptide using the peptide design model based on the feature of the pocket of the training target protein and the first multimodal features of amino acids at positions other than the preset position in the training peptide;

The specific implementation method is the same as step S203 in the embodiment shown in FIG. 2, details can refer to the relevant descriptions in the above embodiment, which will not be repeated here.

S504: Constructing a first loss function based on the second multimodal features of the unnatural amino acid at the preset position and the first multimodal features; For example, when implementing this step specifically, it may include the following steps:
(a1) Obtaining a backbone orientation feature difference, a backbone rotation feature difference, and a side chain type feature difference based on the second multimodal features of the unnatural amino acid at the preset position and the first multimodal features;

The backbone orientation feature difference can take the difference between the backbone orientation feature of the unnatural amino acid at the preset position in the second multimodal features and the backbone orientation feature of the unnatural amino acid at the preset position in the first multimodal features, for example, it can be represented in the form of vector difference.

Correspondingly, the backbone rotation feature difference is similar to the way the backbone orientation feature difference is calculated.

For the side chain type feature difference, since the first multimodal features correspond to the training peptide, the probability of the amino acid type at the preset position in the first multimodal features being an unnatural amino acid can be considered as 1. The second multimodal features are the predicted multimodal features of the unnatural amino acid at the preset position with masking the unnatural amino acid at the preset position, where the probability of the amino acid type at the preset position being an unnatural amino acid is p, where p is greater than 0 and less than 1. In this case, the side chain type feature difference may be 1-p.
(b1) Constructing the first loss function based on the backbone orientation feature difference, the backbone rotation feature difference, and the side chain type feature difference.

Specifically, the first loss function can be constructed as a weighted sum of the backbone orientation feature difference, the backbone rotation feature difference, and the side chain type feature difference. The weights corresponding to each feature can be set based on experience or requirements, which is not limited here.
S505: Adjusting parameters of the peptide design model aiming at convergence of the first loss function.

In this embodiment, supervised training of the peptide design model is exemplified using one set of training data containing a pocket of a training target protein and a training peptide. In practical applications, multiple set of similar training data can be used to train the peptide design model using the above training method until either the number of training iterations reaches a preset threshold or the first loss function consistently converges over multiple consecutive training rounds, at which training terminates and the peptide design model is obtained.

The training method for the peptide design model in this embodiment effectively improves the accuracy of the trained peptide design model by using verified training peptides as positive samples and training the model through supervised learning.

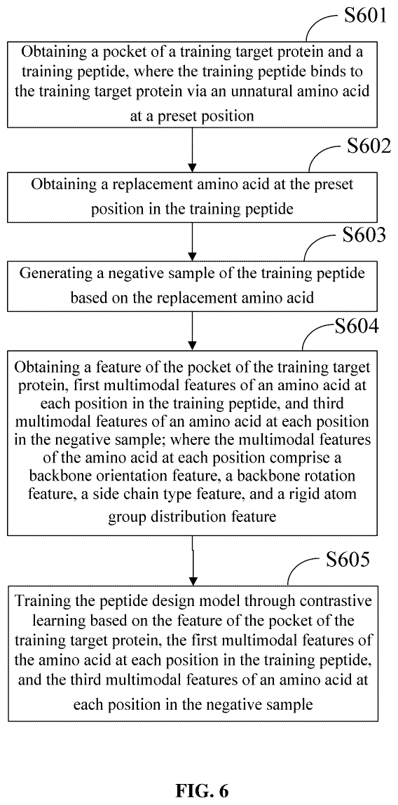

FIG. 6 is a schematic diagram according to a fifth embodiment of the present disclosure. The training method of the peptide design model of this embodiment, based on the technical solution of the embodiment shown in the above FIG. 4, further describes the technical solution of the present disclosure in more detail. As shown in FIG. 6, the training method specifically includes the following steps:
S601: Obtaining a pocket of a training target protein and a training peptide, where the training peptide binds to the training target protein via an unnatural amino acid at a preset position;
S602: Obtaining a replacement amino acid at the preset position in the training peptide;

For example, performing reverse modification on the unnatural amino acid at the preset position in the training peptide to obtain the replacement amino acid; or obtaining an objective amino acid having minimum similarity with the unnatural amino acid at the preset position from a pre-constructed amino acid similarity matrix as the replacement amino acid.

For example, a negative sample with specific physicochemical property differences are generated by reverse modification of the side chain conformations of positive samples from verified peptide sequences, such as mirror-flipping of charged group directions, effectively solving the problem of scarce experimental data for unnatural amino acids.

In this embodiment, the number of rows and columns in the pre-constructed amino acid similarity matrix can equal the sum of the number of natural amino acids and verified unnatural amino acids; each row and column corresponds to one amino acid. The value at each position (i,j) in the matrix equals the similarity between the two amino acids corresponding to that position's row and column; the similarity between two amino acids equals the weighted sum of their structural similarity and pharmacophore distribution similarity. The structural similarity and pharmacophore distribution similarity can be calculated using known experimental methods or pre-trained neural network models.

Since the replacement amino acid selected in this embodiment is used to generate a negative sample, the process involves first locating the row corresponding to the unnatural amino acid at the preset position in the amino acid similarity matrix, then obtaining the objective amino acid corresponding to the column with minimum similarity as the replacement amino acid.

In this embodiment, this method enables side chain replacement while maintaining backbone conformation stability, thereby generating accurate replacement amino acids.
S603: Generating a negative sample of the training peptide based on the replacement amino acid;

Specifically, the replacing includes replacing the unnatural amino acid at the preset position in the training peptide with the replacement amino acid as a negative sample of the training peptide. The replacement amino acid can be either a natural or unnatural amino acid. Since the replacement amino acid is obtained through reverse modification or as the amino acid with minimum similarity, the negative sample is an invalid peptide compared to the objective peptide.
S604: Obtaining a feature of the pocket of the training target protein, first multimodal features of an amino acid at each position in the training peptide, and third multimodal features of an amino acid at each position in the negative sample; where the multimodal features of the amino acid at each position include a backbone orientation feature, a backbone rotation feature, a side chain type feature, and a rigid atom group distribution feature;

The specific implementation can refer to step S102 in the embodiment shown in FIG. 1 or step S202 in the embodiment shown in FIG. 2, which will not be repeated here.
S605: Training the peptide design model through contrastive learning based on the feature of the pocket of the training target protein, the first multimodal features of the amino acid at each position in the training peptide, and the third multimodal features of an amino acid at each position in the negative sample.

For example, step S605 may specifically include the following steps:
(a2) Predicting first predicted multimodal features of the unnatural amino acid at the preset position in the training peptide using the peptide design model based on the feature of the pocket of the training target protein and the first multimodal features of amino acids at positions other than the preset position in the training peptide;
(b2) Predicting second predicted multimodal features of the replacement amino acid at the preset position in the negative sample using the peptide design model based on the feature of the pocket of the training target protein and the third multimodal features of amino acids at positions other than the preset position in the negative sample;
(c2) Adjusting parameters of the peptide design model so that the first predicted probability of the type at the preset position in the first predicted multimodal features being the unnatural amino acid is greater than the second predicted probability of the type at the preset position in the second predicted multimodal features being the replacement amino acid.

In this embodiment, the training peptide is a verified peptide and is used as a positive sample. In practical applications, due to the small number of positive samples, the supervised training method shown in FIG. 5 may not achieve ideal training effects. To improve training effectiveness, the embodiment constructs more negative samples based on positive samples, thereby generating more positive-negative sample pairs.

This allows training the peptide design model through contrastive learning using positive-negative sample pairs.

The training method for the peptide design model in this embodiment can effectively improve the accuracy of the trained peptide design model by training through contrastive learning, as this training method can construct a rich number of positive-negative sample pairs for effective model training.

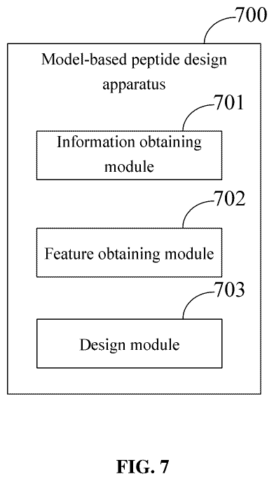

FIG. 7 is a schematic diagram according to a sixth embodiment of the present disclosure. This embodiment provides a model-based peptide design apparatus 700, which includes:
An information obtaining module 701, configured to obtain a pocket of an objective target protein and an objective peptide, where a reserved position for designing an unnatural amino acid is identified in the objective peptide; where the pocket of the objective target protein binds to the objective peptide via the reserved position;
A feature obtaining module 702, configured to obtain a feature of the pocket of the objective target protein and multimodal features of each known amino acid in the objective peptide; where the multimodal features of each known amino acid includes a backbone orientation feature, a backbone rotation feature, a side chain type feature, and a rigid atom group distribution feature;
A design module 703, configured to design the unnatural amino acid at the reserved position in the objective peptide using a pre-trained peptide design model based on the feature of the pocket of the objective target protein and the multimodal features of each known amino acid in the objective peptide.

The implementation principle and technical effects of the model-based peptide design apparatus 700 in this embodiment are the same as those in the above-related method embodiments, which can be referred to in detail in the above-related method embodiments and will not be repeated here.

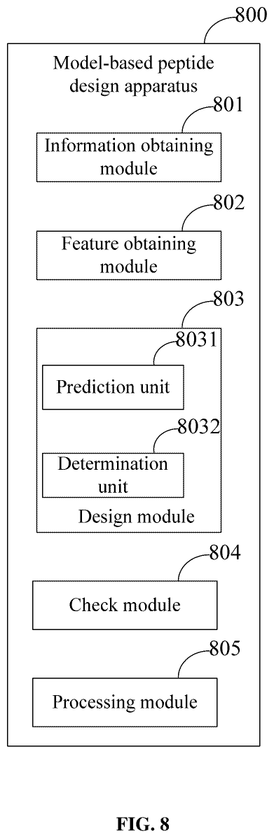

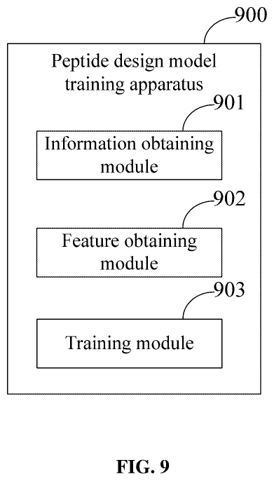

FIG. 8 is a schematic diagram according to a seventh embodiment of the present disclosure. The model-based peptide design apparatus 800 in this embodiment further describes the technical solution in more detail based on the technical solution of the embodiment shown in FIG. 7. As shown in FIG. 8, the model-based peptide design apparatus 800 includes modules with the same names and functions as shown in FIG. 7: an information obtaining module 801, a feature obtaining module 802, and a design module 803.

The design module 803 includes:
A prediction unit 8031, configured to predict multimodal features of the unnatural amino acid at the reserved position in the objective peptide using the peptide design model based on the feature of the pocket of the objective target protein and the multimodal features of each known amino acid in the objective peptide;
A determination unit 8032, configured to determine the structure of the unnatural amino acid at the reserved position in the objective peptide based on the multimodal features of the unnatural amino acid.

Optionally further, in one embodiment of the present disclosure, the prediction unit 8031 is configured to:
predict features of the unnatural amino acid at the reserved position in the objective peptide using the peptide design model by referring to pre-obtained initial features of the unnatural amino acid at the reserved position, based on the feature of the pocket of the objective target protein and the multimodal features of each known amino acid in the objective peptide.

Optionally further, in one embodiment of the present disclosure, the feature obtaining module 802 is configured to:
obtain an initial backbone orientation feature of the unnatural amino acid at the reserved position in the objective peptide based on a Gaussian distribution;
obtain an initial backbone rotation feature of the unnatural amino acid at the reserved position in the objective peptide based on a uniform distribution;
obtain an initial side chain type feature of the unnatural amino acid at the reserved position in the objective peptide based on a Gaussian distribution; and
obtain an initial rigid atom group distribution feature of the unnatural amino acid at the reserved position in the objective peptide based on a uniform distribution.

Optionally further, as shown in FIG. 8, in one embodiment of the present disclosure, the peptide design apparatus 800 for designing the unnatural amino acid in the peptide also includes:

A check module 804, configured to perform validity check based on a structure of the pocket of the objective target protein, a structure of each known amino acid in the objective peptide, and a structure of the unnatural amino acid at the reserved position; and

A processing module 805, configured to discard the designed unnatural amino acid for the reserved position upon validation failure.

The implementation principle and technical effects of the model-based peptide design apparatus 800 in this embodiment are the same as those in the above-related method embodiments, which can be referred to in detail in the above-related method embodiments and will not be repeated here.

FIG. 9 is a schematic diagram according to a eighth embodiment of the present disclosure.

This embodiment provides a peptide design model training apparatus 900, which includes:
An information obtaining module 901, configured to obtain a pocket of a training target protein and a training peptide, where the training peptide binds to the training target protein via an unnatural amino acid at a preset position;
A feature obtaining module 902, configured to obtain a feature of the pocket of the training target protein and first multimodal features of an amino acid at each position in the training peptide; where the first multimodal features of the amino acid at each position include a backbone orientation feature, a backbone rotation feature, a side chain type feature, and a rigid atom group distribution feature;
A training module 903, configured to train the peptide design model based on the feature of the pocket of the training target protein and the first multimodal features of the amino acid at each position in the training peptide.

The implementation principle and technical effects of the peptide design model training apparatus 900 in this embodiment are the same as those in the above-related method embodiments, which can be referred to in detail in the above-related method embodiments and will not be repeated here.

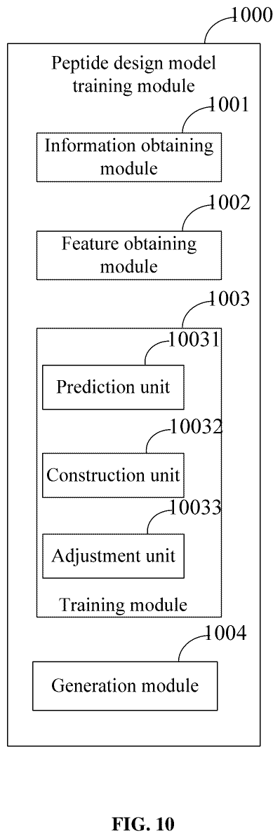

FIG. 10 is a schematic diagram according to a ninth embodiment of the present disclosure.

The peptide design model training apparatus 1000 of the embodiment further describes the technical solution of the present application in more detail based on the technical solution of the embodiment shown in FIG. 9. As shown in FIG. 10, the peptide design model training apparatus 1000 of the embodiment includes modules with same names and same functions as shown in FIG. 9: an information obtaining module 1001, a feature obtaining module 1002, and a training module 1003. As shown in FIG. 10, in the embodiment, the training module 1003 includes:
A prediction unit 10031, configured to predict second multimodal features of the unnatural amino acid at the preset position in the training peptide using the peptide design model based on the feature of the pocket of the training target protein and the first multimodal features of amino acids at positions other than the preset position in the training peptide;
A construction unit 10032, configured to construct a first loss function based on the second multimodal features of the unnatural amino acid at the preset position and the first multimodal features;
An adjustment unit 10033, configured to adjust parameters of the peptide design model aiming at convergence of the first loss function.

Optionally further, in one embodiment of the present disclosure, the construction unit 10032 is configured to:
obtain a backbone orientation feature difference, a backbone rotation feature difference, and a side chain type feature difference based on the second multimodal features of the unnatural amino acid at the preset position and the first multimodal features; and
construct the first loss function based on the backbone orientation feature difference, the backbone rotation feature difference, and the side chain type feature difference.

Optionally further, as shown in FIG. 10, the training apparatus 1000 also includes a generation module 1004:
An information obtaining module 1001, configured to obtain a replacement amino acid at the preset position in the training peptide; and
A generation module 1004, configured to generate a negative sample of the training peptide based on the replacement amino acid.

Optionally further, in one embodiment of the present disclosure, the training module 1003 is configured to:
train the peptide design model through contrastive learning based on the feature of the pocket of the training target protein, the first multimodal features of the amino acid at each position in the training peptide, and third multimodal features of an amino acid at each position in the negative sample.

Optionally further, in one embodiment of the present disclosure, the information obtaining module 1001 is configured to:
perform reverse modification on the unnatural amino acid at the preset position in the training peptide to obtain the replacement amino acid;
or
obtain an objective amino acid having minimum similarity with the unnatural amino acid at the preset position from a pre-constructed amino acid similarity matrix as the replacement amino acid.

Optionally further, in one embodiment of the present disclosure, the training module 1003 is configured to:
predict first predicted multimodal features of the unnatural amino acid at the preset position in the training peptide using the peptide design model based on the feature of the pocket of the training target protein and the first multimodal features of amino acids at positions other than the preset position in the training peptide;
predict second predicted multimodal features of the replacement amino acid at the preset position in the negative sample using the peptide design model based on the feature of the pocket of the training target protein and the third multimodal features of amino acids at positions other than the preset position in the negative sample; and
adjust parameters of the peptide design model so that the first predicted probability of the type at the preset position in the first predicted multimodal features being the unnatural amino acid is greater than the second predicted probability of the type at the preset position in the second predicted multimodal features being the replacement amino acid.

The implementation principle and technical effects of the training apparatus 1000 in this embodiment are the same as those in the above-related method embodiments, which can be referred to in detail in the above-related method embodiments and will not be repeated here.

In the technical solution of the present disclosure, the acquisition, storage, and application of user personal information comply with relevant laws and regulations and do not violate public order and good morals.

According to embodiments of the present disclosure, the present disclosure also provides an electronic device, a readable storage medium, and a computer program product.

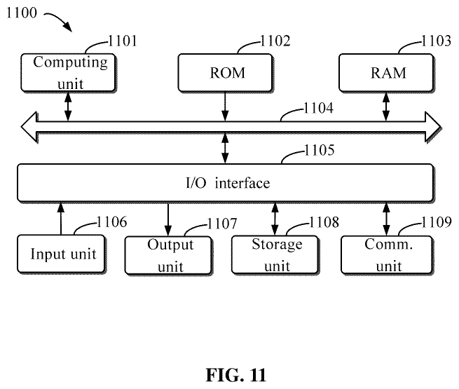

FIG. 11 shows a schematic block diagram of an electronic device 1100 which may be configured to implement the embodiment of the present disclosure. The electronic device is intended to represent various forms of digital computers, such as laptop computers, desktop computers, workstations, servers, blade servers, mainframe computers, and other appropriate computers. The electronic device may also represent various forms of mobile apparatuses, such as personal digital assistants, cellular telephones, smart phones, wearable devices, and other similar computing apparatuses. The components shown herein, their connections and relationships, and their functions, are meant to be exemplary only, and are not meant to limit implementation of the present disclosure described and/or claimed herein.

As shown in FIG. 11, the device 1100 includes a computing unit 1101 which may perform various appropriate actions and processing operations according to a computer program stored in a read only memory (ROM) 1102 or a computer program loaded from a storage unit 1108 into a random access memory (RAM) 1103. Various programs and data necessary for the operation of the device 1100 may be also stored in the RAM 1103. The computing unit 1101, the ROM 1102, and the RAM 1103 are connected with one other through a bus 1104. An input/output (I/O) interface 1105 is also connected to the bus 1104.

The plural components in the device 1100 are connected to the I/O interface 1105, and include: an input unit 1106, such as a keyboard, a mouse, or the like; an output unit 1107, such as various types of displays, speakers, or the like; the storage unit 1108, such as a magnetic disk, an optical disk, or the like; and a communication unit (comm. unit) 1109, such as a network card, a modem, a wireless communication transceiver, or the like. The communication unit 1109 allows the device 1100 to exchange information/data with other devices through a computer network, such as the Internet, and/or various telecommunication networks.

The computing unit 1101 may be a variety of general and/or special purpose processing components with processing and computing capabilities. Some examples of the computing unit 1101 include, but are not limited to, a central processing unit (CPU), a graphic processing unit (GPU), various dedicated artificial intelligence (AI) computing chips, various computing units running machine learning model algorithms, a digital signal processor (DSP), and any suitable processor, controller, microcontroller, or the like. The computing unit 1101 performs the methods and processing operations described above, such as the method according to the present disclosure. For example, in some embodiments, the method according to the present disclosure may be implemented as a computer software program tangibly contained in a machine readable medium, such as the storage unit 1108. In some embodiments, part or all of the computer program may be loaded and/or installed into the device 1100 via the ROM 1102 and/or the communication unit 1109. When the computer program is loaded into the RAM 1103 and executed by the computing unit 1101, one or more steps of the method according to the present disclosure may be performed. Alternatively, in other embodiments, the computing unit 1101 may be configured to perform the method according to the present disclosure by any other suitable means (for example, by means of firmware).

Various implementations of the systems and technologies described herein above may be implemented in digital electronic circuitry, integrated circuitry, field programmable gate arrays (FPGA), application specific integrated circuits (ASIC), application specific standard products (ASSP), systems on chips (SOC), complex programmable logic devices (CPLD), computer hardware, firmware, software, and/or combinations thereof. The systems and technologies may be implemented in one or more computer programs which are executable and/or interpretable on a programmable system including at least one programmable processor, and the programmable processor may be special or general, and may receive data and instructions from, and transmit data and instructions to, a storage system, at least one input apparatus, and at least one output apparatus.

Program codes for implementing the method according to the present disclosure may be written in any combination of one or more programming languages. These program codes may be provided to a processor or a controller of a general purpose computer, a special purpose computer, or other programmable data processing apparatuses, such that the program code, when executed by the processor or the controller, causes functions/operations specified in the flowchart and/or the block diagram to be implemented. The program code may be executed entirely on a machine, partly on a machine, partly on a machine as a stand-alone software package and partly on a remote machine, or entirely on a remote machine or a server.

In the context of the present disclosure, the machine readable medium may be a tangible medium which may contain or store a program for use by or in connection with an instruction execution system, apparatus, or device. The machine readable medium may be a machine readable signal medium or a machine readable storage medium. The machine readable medium may include, but is not limited to, an electronic, magnetic, optical, electromagnetic, infrared, or semiconductor system, apparatus, or device, or any suitable combination of the foregoing. More specific examples of the machine readable storage medium may include an electrical connection based on one or more wires, a portable computer disk, a hard disk, a random access memory (RAM), a read only memory (ROM), an erasable programmable read only memory (EPROM or flash memory), an optical fiber, a portable compact disc read only memory (CD-ROM), an optical storage device, a magnetic storage device, or any suitable combination of the foregoing.

To provide interaction with a user, the systems and technologies described here may be implemented on a computer having: a display apparatus (for example, a cathode ray tube (CRT) or liquid crystal display (LCD) monitor) for displaying information to a user; and a keyboard and a pointing apparatus (for example, a mouse or a trackball) by which a user may provide input for the computer. Other kinds of apparatuses may also be used to provide interaction with a user; for example, feedback provided for a user may be any form of sensory feedback (for example, visual feedback, auditory feedback, or tactile feedback); and input from a user may be received in any form (including acoustic, speech or tactile input).

The systems and technologies described here may be implemented in a computing system (for example, as a data server) which includes a back-end component, or a computing system (for example, an application server) which includes a middleware component, or a computing system (for example, a user computer having a graphical user interface or a web browser through which a user may interact with an implementation of the systems and technologies described here) which includes a front-end component, or a computing system which includes any combination of such back-end, middleware, or front-end components. The components of the system may be interconnected through any form or medium of digital data communication (for example, a communication network). Examples of the communication network include: a local area network (LAN), a wide area network (WAN) and the Internet.

A computer system may include a client and a server. Generally, the client and the server are remote from each other and interact through the communication network. The relationship between the client and the server is generated by virtue of computer programs which run on respective computers and have a client-server relationship to each other. The server may be a cloud server or a server of a distributed system, or a server incorporating a blockchain.

It should be understood that various forms of the flows shown above may be used and reordered, and steps may be added or deleted. For example, the steps described in the present disclosure may be executed in parallel, sequentially, or in different orders, which is not limited herein as long as the desired results of the technical solution disclosed in the present disclosure may be achieved.

The above-mentioned implementations are not intended to limit the scope of the present disclosure. It should be understood by those skilled in the art that various modifications, combinations, sub-combinations and substitutions may be made, depending on design requirements and other factors. Any modification, equivalent substitution and improvement made within the spirit and principle of the present disclosure all should be included in the extent of protection of the present disclosure.

What is claimed is:

1. A model-based peptide design method, comprising:
obtaining a pocket of an objective target protein and an objective peptide, wherein a reserved position for designing an unnatural amino acid is identified in the objective peptide; wherein the pocket of the objective target protein binds to the objective peptide via the reserved position;
obtaining a feature of the pocket of the objective target protein and multimodal features of each known amino acid in the objective peptide; wherein the multimodal features of each known amino acid comprise a backbone orientation feature, a backbone rotation feature, a side chain type feature, and a rigid atom group distribution feature; and
designing the unnatural amino acid at the reserved position in the objective peptide using a peptide design model based on the feature of the pocket of the objective target protein and the multimodal features of each known amino acid in the objective peptide.

2. The method according to claim 1, wherein designing the unnatural amino acid at the reserved position in the objective peptide using the pre-trained peptide design model based on the feature of the pocket of the objective target protein and the multimodal features of each known amino acid in the objective peptide comprises:
predicting multimodal features of the unnatural amino acid at the reserved position in the objective peptide using the peptide design model based on the feature of the pocket of the objective target protein and the multimodal features of each known amino acid in the objective peptide; and
determining a structure of the unnatural amino acid at the reserved position in the objective peptide based on the multimodal features of the unnatural amino acid.

3. The method according to claim 2, wherein predicting the multimodal features of the unnatural amino acid at the reserved position in the objective peptide using the peptide design model based on the feature of the pocket of the objective target protein and the multimodal features of each known amino acid in the objective peptide comprises:
predicting features of the unnatural amino acid at the reserved position in the objective peptide using the peptide design model by referring to pre-obtained initial features of the unnatural amino acid at the reserved position, based on the feature of the pocket of the objective target protein and the multimodal features of each known amino acid in the objective peptide.

4. The method according to claim 3, wherein before predicting the features of the unnatural amino acid at the reserved position in the objective peptide using the peptide design model by referring to the pre-obtained initial features based on the feature of the pocket of the objective target protein and the multimodal features of each known amino acid in the objective peptide, the method further comprises:
obtaining an initial backbone orientation feature of the unnatural amino acid at the reserved position in the objective peptide based on a Gaussian distribution;
obtaining an initial backbone rotation feature of the unnatural amino acid at the reserved position in the objective peptide based on a uniform distribution;
obtaining an initial side chain type feature of the unnatural amino acid at the reserved position in the objective peptide based on a Gaussian distribution; and
obtaining an initial rigid atom group distribution feature of the unnatural amino acid at the reserved position in the objective peptide based on a uniform distribution.

5. The method according to claim 2, wherein after designing the unnatural amino acid at the reserved position in the objective peptide using the pre-trained peptide design model based on the feature of the pocket of the objective target protein and the multimodal features of each known amino acid in the objective peptide, the method further comprises:
performing a validity check based on a structure of the pocket of the objective target protein, a structure of each known amino acid in the objective peptide, and a structure of the unnatural amino acid at the reserved position; and
discarding the designed unnatural amino acid for the reserved position upon validation failure.

6. The method according to claim 1, wherein the peptide design model is trained by:
obtaining a pocket of a training target protein and a training peptide, wherein the training peptide binds to the training target protein via an unnatural amino acid at a preset position;
obtaining a feature of the pocket of the training target protein and first multimodal features of an amino acid at each position in the training peptide; wherein the first multimodal features of the amino acid at each position comprise a backbone orientation feature, a backbone rotation feature, a side chain type feature, and a rigid atom group distribution feature;
training the peptide design model based on the feature of the pocket of the training target protein and the first multimodal features of the amino acid at each position in the training peptide.

7. The method according to claim 6, wherein training the peptide design model based on the feature of the pocket of the training target protein and the first multimodal features of the amino acid at each position in the training peptide comprises:
predicting second multimodal features of the unnatural amino acid at the preset position in the training peptide using the peptide design model based on the feature of the pocket of the training target protein and the first multimodal features of amino acids at positions other than the preset position in the training peptide;
constructing a first loss function based on the second multimodal features of the unnatural amino acid at the preset position and the first multimodal features; and
adjusting parameters of the peptide design model aiming at convergence of the first loss function.

8. The method according to claim 7, wherein constructing the first loss function based on the second multimodal features of the unnatural amino acid at the preset position and the first multimodal features comprises:
obtaining a backbone orientation feature difference, a backbone rotation feature difference, and a side chain type feature difference based on the second multimodal features of the unnatural amino acid at the preset position and the first multimodal features; and
constructing the first loss function based on the backbone orientation feature difference, the backbone rotation feature difference, and the side chain type feature difference.

9. The method according to claim 6, wherein before training the peptide design model based on the feature of the pocket of the training target protein and the first multimodal features of the amino acid at each position in the training peptide, the method further comprises:
obtaining a replacement amino acid at the preset position in the training peptide; and
generating a negative sample of the training peptide based on the replacement amino acid.

10. The method according to claim 9, wherein training the peptide design model based on the feature of the pocket of the training target protein and the first multimodal features of the amino acid at each position in the training peptide comprises:
training the peptide design model through contrastive learning based on the feature of the pocket of the training target protein, the first multimodal features of the amino acid at each position in the training peptide, and third multimodal features of an amino acid at each position in the negative sample.

11. The method according to claim 9, wherein obtaining the replacement amino acid at the preset position in the training peptide comprises:
performing reverse modification on the unnatural amino acid at the preset position in the training peptide to obtain the replacement amino acid.

12. The method according to claim 9, wherein obtaining the replacement amino acid at the preset position in the training peptide comprises:
obtaining an objective amino acid having minimum similarity with the unnatural amino acid at the preset position from a pre-constructed amino acid similarity matrix as the replacement amino acid.

13. The method according to claim 10, wherein training the peptide design model through contrastive learning based on the feature of the pocket of the training target protein, the first multimodal features of the amino acid at each position in the training peptide, and the third multimodal features of the amino acid at each position in the negative sample comprises:
predicting first predicted multimodal features of the unnatural amino acid at the preset position in the training peptide using the peptide design model based on the feature of the pocket of the training target protein and the first multimodal features of amino acids at positions other than the preset position in the training peptide;
predicting second predicted multimodal features of the replacement amino acid at the preset position in the negative sample using the peptide design model based on the feature of the pocket of the training target protein and the third multimodal features of amino acids at positions other than the preset position in the negative sample; and
adjusting parameters of the peptide design model so that a first predicted probability of the type at the preset position in the first predicted multimodal features being the unnatural amino acid is greater than a second predicted probability of the type at the preset position in the second predicted multimodal features being the replacement amino acid.

14. An electronic device, comprising:
at least one processor; and
a memory communicatively connected to the at least one processor; wherein
the memory stores instructions executable by the at least one processor to cause the at least one processor to perform a model-based peptide design method, which comprises:
obtaining a pocket of an objective target protein and an objective peptide, wherein a reserved position for designing an unnatural amino acid is identified in the objective peptide; wherein the pocket of the objective target protein binds to the objective peptide via the reserved position;
obtaining a feature of the pocket of the objective target protein and multimodal features of each known amino acid in the objective peptide; wherein the multimodal features of each known amino acid comprise a backbone orientation feature, a backbone rotation feature, a side chain type feature, and a rigid atom group distribution feature; and
designing the unnatural amino acid at the reserved position in the objective peptide using a peptide design model based on the feature of the pocket of the objective target protein and the multimodal features of each known amino acid in the objective peptide.

15. The electronic device according to claim 14, wherein designing the unnatural amino acid at the reserved position in the objective peptide using the pre-trained peptide design model based on the feature of the pocket of the objective target protein and the multimodal features of each known amino acid in the objective peptide comprises:
predicting multimodal features of the unnatural amino acid at the reserved position in the objective peptide using the peptide design model based on the feature of the pocket of the objective target protein and the multimodal features of each known amino acid in the objective peptide; and
determining a structure of the unnatural amino acid at the reserved position in the objective peptide based on the multimodal features of the unnatural amino acid.

16. The electronic device according to claim 15, wherein predicting the multimodal features of the unnatural amino acid at the reserved position in the objective peptide using the peptide design model based on the feature of the pocket of the objective target protein and the multimodal features of each known amino acid in the objective peptide comprises:
predicting features of the unnatural amino acid at the reserved position in the objective peptide using the peptide design model by referring to pre-obtained initial features of the unnatural amino acid at the reserved position, based on the feature of the pocket of the objective target protein and the multimodal features of each known amino acid in the objective peptide.

17. The electronic device according to claim 14, wherein the peptide design model is trained by:
obtaining a pocket of a training target protein and a training peptide, wherein the training peptide binds to the training target protein via an unnatural amino acid at a preset position;
obtaining a feature of the pocket of the training target protein and first multimodal features of an amino acid at each position in the training peptide; wherein the first multimodal features of the amino acid at each position comprise a backbone orientation feature, a backbone rotation feature, a side chain type feature, and a rigid atom group distribution feature;
training the peptide design model based on the feature of the pocket of the training target protein and the first multimodal features of the amino acid at each position in the training peptide.

18. The electronic device according to claim 17, wherein training the peptide design model based on the feature of the pocket of the training target protein and the first multimodal features of the amino acid at each position in the training peptide comprises:
predicting second multimodal features of the unnatural amino acid at the preset position in the training peptide using the peptide design model based on the feature of the pocket of the training target protein and the first multimodal features of amino acids at positions other than the preset position in the training peptide;
constructing a first loss function based on the second multimodal features of the unnatural amino acid at the preset position and the first multimodal features; and
adjusting parameters of the peptide design model aiming at convergence of the first loss function.

19. The electronic device according to claim 18, wherein constructing the first loss function based on the second multimodal features of the unnatural amino acid at the preset position and the first multimodal features comprises:
obtaining a backbone orientation feature difference, a backbone rotation feature difference, and a side chain type feature difference based on the second multimodal features of the unnatural amino acid at the preset position and the first multimodal features; and
constructing the first loss function based on the backbone orientation feature difference, the backbone rotation feature difference, and the side chain type feature difference.

20. A non-transitory computer-readable storage medium storing computer instructions for causing a computer to perform a model-based peptide design method, which comprises:
obtaining a pocket of an objective target protein and an objective peptide, wherein a reserved position for designing an unnatural amino acid is identified in the objective peptide; wherein the pocket of the objective target protein binds to the objective peptide via the reserved position;
obtaining a feature of the pocket of the objective target protein and multimodal features of each known amino acid in the objective peptide; wherein the multimodal features of each known amino acid comprise a backbone orientation feature, a backbone rotation feature, a side chain type feature, and a rigid atom group distribution feature; and
designing the unnatural amino acid at the reserved position in the objective peptide using a peptide design model based on the feature of the pocket of the objective target protein and the multimodal features of each known amino acid in the objective peptide.
